(19) United States
(12) Patent Application Publication
(10) Pub. No.: US 2026/0207118 A1
(43) Pub. Date: Jul. 23, 2026

(54) DEVICE AND METHOD FOR MEASURING ELECTROMYOGRAPHIC SIGNALS INDICATIVE OF RESPIRATORY EFFORT WITH A WEARABLE DEVICE ON A SINGLE LIMB

(71) Applicant: The Research Foundation for the State University of New York, Albany, NY (US)

(72) Inventors: Mark Stewart, Brooklyn, NY (US); Rena Orman, Brooklyn, NY (US)

(21) Appl. No.: 19/131,786

(22) PCT Filed: Nov. 17, 2023
(86) PCT No.: PCT/US2023/080250

§ 371 Date: May 21, 2025

Related U.S. Application Data
(60) Provisional application No. 63/426,971, filed on Nov. 21, 2022.

(51) Int. Cl. A61B 5/389; A61B 5/00; A61B 5/08; A61B 5/256

(52) U.S. Cl. CPC A61B 5/389; A61B 5/0826; A61B 5/256; A61B 5/4094; A61B 5/681

(57) ABSTRACT

A device and method for monitoring electromyographic signals at a wearable health-data device that selectively monitors bioelectrical signals of a human body on a single limb. The device, such as a smart-watch or bracelet, has a body with a surface having at least two electrical contacts that are placed apart from each other and conductively held against the human body such that a bioelectrical electrical signal is passed between the two electrical contacts. There can be a computer platform contained within or on the device body that selectively monitors bioelectrical signals, isolates one or more electromyographic signals within the monitored bioelectrical signals, compiles respiratory effort data from the one or more electromyographic signals, and selectively outputs the respiratory effort data. An existing health-data device, with one body-facing contact, can also be modified with a fitting that allows dual contacts to be simultaneously held against the human body.

### CROSS-REFERENCE TO RELATED APPLICATION

This application claims the benefit of U.S. Provisional Patent Application No. 63/426,971, filed on Nov. 21, 2022, the entirety of which is hereby incorporated herein by this reference.

### BACKGROUND OF THE INVENTION

### 1. Field of the Invention

The present invention relates to medical devices that monitor bioelectrical signals within humans. More specifically, the present invention relates to monitoring electromyographic signals indicative of respiratory effort at a single wearable device on a single limb or single location on the body.

### 2. Description of the Related Art

Electromyographic (EMG) signals are electrical signals from muscles. With many recording configurations intended to detect electrocardiogram (ECG) signals, EMG signals can be simultaneously detected. EMG signals originating from the muscles of respiration are associated with and proportional to respiratory effort. The EMG signal comes in part from the muscles of the thoracic cavity and can be seen in ECG recordings with sufficient bandpass. When at least two monitoring electrodes are placed close to each other on the human body, such as on the same arm (or wrist), the differential nature of the recordings eliminates the ECG as a major component of the bioelectrical signal but leaves intact a majority of the respiratory effort-associated EMG signal.

Several adverse medical events can cause or be a consequence of respiratory distress. For example, ictal apnea (a cause of respiratory distress) and/or ictal bradycardia (a consequence of respiratory distress) has been demonstrated to occur during some epileptic seizures and contribute to sudden death in epilepsy (SUDEP). SUDEP is the major cause of death among persons with epilepsy. One suspected cause of ictal respiratory distress is laryngospasm, a tonic adduction of the vocal folds that partially or fully obstructs the upper airway producing a period of obstructive apnea. The respiratory distress from the laryngospasm is detectable as intense respiratory effort during the period of airway obstruction. The monitoring of EMG signals for epileptic events is described in WO2018089789A1, the entirety of which is hereby incorporated herein by this reference.

Another adverse medical event that is indicated by respiratory distress is sleep apnea. Sleep apnea is a potentially serious sleep disorder in which breathing repeatedly stops and starts. The main types of sleep apnea are: obstructive sleep apnea, which occurs when throat muscles relax; central sleep apnea, which occurs when the brain does not properly control the respiratory muscles; and complex sleep apnea syndrome, which is a combination of both obstructive sleep apnea and central sleep apnea. Sleep apnea can have many adverse cumulative physical effects, such as cardiovascular disease, liver function impairment, brain tissue loss, and seizures even in the absence of epilepsy. Even death could occur from untreated sleep apnea due to lack of oxygen to the body.

There has been a proliferation of wearable devices that can monitor bioelectrical signals. The wearable devices can include a Bluetooth heart monitor worn in “sports bra,” and other bands and sleeves worn on the body that include a wireless biostatistics monitor. One category of these devices is generally referred to as “smartwatches.” Smartwatches have been increasingly equipped with hardware and software capabilities to make them practical biosensors. As an example, recent Apple watches enable electrocardiogram (ECG) recordings for the evaluation of heart rate and rhythm. These electrical signal recordings are made with a pair of electrical contacts on the surface of the body of the watch. One electrode is on the watch back and is in continuous contact with the skin of the arm that is wearing the watch. The second electrode is on the surface of the watch crown and is intended for contacting the tip of a finger from the opposite arm. This configuration puts one electrode on each arm and approximates the conventional “limb lead” configuration for clinical ECG recordings used to evaluate heart rate and rhythm.

EMG signals intended for assessing respiratory effort, however, can benefit from both electrodes being placed proximate to each other, such as on the same limb or very near each other on the torso. The differential nature of the recordings eliminates the ECG as a major electrical “contaminant” of the electrical signal but leaves intact a majority of the respiratory effort-associated EMG signal. Other wearable bioelectrical signal-detecting devices have great difficulty in detecting EMG signals within ECG signals. Additionally, since extant smart watches are intended to capture ECG signals, these devices intentionally filter the EMG signals normally co-recorded with the ECG to provide a clean ECG signal.

It is accordingly to the problem of monitoring electromyographic signals indicative of respiratory effort with a single wearable device at a single location on the body, such as a single limb, that the present invention is primarily directed.

### BRIEF SUMMARY OF THE INVENTION

Briefly described, the present invention includes a device and method for monitoring electromyographic signals at a wearable health-data device that selectively monitors bioelectrical signals of a human body, such as a smart-watch or other wireless biosensor, from a single point or limb. An existing health-data device, with one body-facing contact, can also be modified with a fitting that allows dual contacts to be simultaneously held against the human body such that electromyographic signals can be monitored from a single limb or point on the body.

Furthermore, existing devices intended to capture ECG, including smart watches, actively filter as much non-ECG signal as possible for the cleanest ECG possible. And for an ECG signal, one needs electrodes on two limbs. With a wearable device, such as a smart watch, this means that the users must be capable of holding their non-watch hand on the watch, which does not allow anyone sleeping, unconscious, disabled, or too young to follow instructions (e.g., infants) to use the device to optimally capture bioelectrical signals. In contrast, the present invention allows these types of users because of the single location of monitoring.

In one embodiment, the invention includes a device for monitoring electromyographic signals, with a device body having a surface thereof that is configured to be held against a human body. There are two electrical contacts on the surface of the device body, with each contact placed apart from the other on the device body such that each contact is conductively held against the human body such that a bioelectrical electrical signal is recorded between the two electrical contacts. There is a computer platform contained within the device body that is configured to selectively monitor bioelectrical signals between the electrical contacts on the device body, isolate one or more electromyographic signals within the monitored bioelectrical signals, compile respiratory effort data from the one or more electromyographic signals, and selectively output the respiratory effort data.

The device body can further be embodied as including a transmitter therein, with the transmitter in communication with the computer platform and selectively transmitting the respiratory effort data output from the computer platform. Furthermore, the device body can further include a receiver therein, with the receiver in communication with the computer platform, and in communication with a communication network and selectively receiving data sent thereacross.

The device can also be configured to be attached to a wristband such that the wristband selectively holds the device body and the electrical contacts against a wrist. Alternately, the device can use an elastomeric mechanism or other physical mechanism to hold itself in place against the human body such that the electrical contacts can monitor bioelectrical signals.

Via the computer platform, the device can determine the presence of changes in respiratory effort or periods of obstructive apnea based upon the respiratory effort data based upon the respiratory effort data from the EMG signals that can be obtained from the bioelectrical signals across the two contacts.

In one embodiment, the invention includes a fitting for modifying a wearable health-data device to monitor electromyographic signals, such as a smartwatch or other device that does not have adjacent body-contacting electrodes or electrical contacts such that EMG signals are more easily discerned from ECG signals. The fitting includes a body including an electrical contact and electrical conduit, with a mechanism that allows the selectively attachment of the fitting body to a health-data device that is configured to be selectively held against a human body, where the health-data device includes a first electrical contact that is selectively conductively held against the human body and a second electrical contact that is not conductively held against the human body. The fitting can further be configured to be selectively attached to the body of the health-data device such that the electrical contact of the fitting is conductively held against the human body when the heath-data device is held thereagainst, with the fitting further attached such that a bioelectrical signal is passed from the electrical contact of the fitting and through the electrical conduit to the second electrical contact of the health-data device.

In one embodiment, the device includes a clip that snaps to the face of a smartwatch that has an electrically conductive crown and provides an electrical contact that redirects the watch's crown electrode contact onto the skin of the arm wearing the watch. The electrical contact that touches the crown is electrically connected through the body of the “right-angled” assembly to the electrical contact directed toward the skin surface. This also prevents the watch from being used for two-handed recordings while the accessory is in place. The position of the electrical surface contacting the watch crown can be fixed in relation to the position of the contact that will touch skin, or can be adjusted perpendicularly to the face of the watch to optimize skin contact. In another embodiment, the position of the electrical contact with the crown is fixed, and a flexible (e.g., spring-loaded) contact reaches from the watch accessory to the skin to optimize skin contact.

Alternately, the mechanism for selectively attaching the body to a health-data device can be an elastomeric mechanism. Further, the electrical conduit can be a wire, or the electrical contact and electrical conduit of the fitting are the same conductive component, such as a single conductive element.

In the smartwatch-modified-with-fitting configuration, the invention then includes the device body which has a surface thereof, where the device body configured to be held against a human body, and a first electrical contact that is selectively conductively held against the human body and a second electrical contact that is not conductively held against the human body. The fitting includes a fitting body having a fitting electrical contact and fitting electrical conduit, and a mechanism selectively attaching the fitting body to the device such that the fitting electrical contact is conductively held against the human body and a bioelectrical signal is passed from the fitting electrical contact and through the fitting electrical conduit to the second electrical contact of the device. Included in the device body is a computer platform that is configured to selectively monitor bioelectrical signals between the fitting electrical contact and second electrical contact, isolate one or more electromyographic signals within the monitored bioelectrical signals, compile respiratory effort data from the one or more electromyographic signals, and selectively output the respiratory effort data.

In another embodiment, the device for monitoring electromyographic signals has a flexible device body, the body comprised of a plurality of electrical wires, with at least two conductive electrodes on the body that each have fully conductive exterior surfaces to contact skin continuously, and each electrode having a fully conductive interior surface that contacts the exposed surface at least one of the plurality of electrical wires of the body. The device also has a computer platform on the body, the computer platform configured to selectively monitor bioelectrical signals between the electrodes on the device body, isolate one or more electromyographic signals within the monitored bioelectrical signals, compile respiratory effort data from the one or more electromyographic signals, and selectively transmit the respiratory effort data.

The flexible device body can be made of a braided 3-wire bracelet, each having at least one exposed conductive portion, with the conductive electrodes being three beads with fully conductive interior surfaces that contact at least one exposed surface of each of one of the three wires. The computer platform can include a recording amplifier, Bluetooth transmitter, and power source.

The invention can also include a method for monitoring electromyographic signals at a wearable device holding a device against a human body, the device having a device body with a surface thereof and two electrical contacts on the surface of the device body, with each contact placed apart from the other on the device body such that each contact is conductively held against the human body such that a bioelectrical electrical signal is passed between the two electrical contacts. Then the method includes the steps of selectively monitoring bioelectrical signals between the electrical contacts on the device body, isolating one or more electromyographic signals within the monitored bioelectrical signals, compiling respiratory effort data from the one or more electromyographic signals, and selectively outputting the respiratory effort data.

The present invention thus provides an advantage in that it allows the monitoring of EMG signals at a wearable device at a single location on the human body, such as a limb. The present invention also has industrial applicability in that it provides a fitting body that can modify an existing health-data gathering wearable device, such as a smartwatch, that otherwise is configured to just monitor ECG biometric signals across the body of the wearer. Furthermore, the device can be placed on the limb an unconscious person or infant who are unable to follow directions or assist in the placement of the wearable device or hold separate electrodes to optimize the recording of signals. These and other advantages of the present invention will be apparent to one of skill in the art after review of the full description of the invention below.

### BRIEF DESCRIPTION OF THE DRAWINGS

FIG. 1A is a top perspective view of one embodiment of a wearable device embodied as a smartwatch.

FIG. 1B is a bottom perspective view of the smartwatch of FIG. 1A illustrating two body-contacting electrical contacts that can monitor the bioelectrical signals of the body.

FIG. 2 is a block diagram of the computer platform and components thereon which is within the body of the device illustrated in FIGS. 1A and 1B and 4A and 4B.

FIG. 3A is a top view of one embodiment of a fitting body comprises of an elastomer that modifies a smartwatch which has only one body-facing electrical contact

FIG. 3B is a bottom view of the fitting body of FIG. 3A, illustrating the electrical contact and electrical conduit.

FIG. 3C is a sideview of the fitting body of FIGS. 3A and 3B.

FIG. 4A is a top perspective view of a smartwatch having a conductive crown.

FIG. 4B is a bottom perspective view of the smartwatch of FIG. 4A illustrating a single body-facing contact.

FIG. 4C is a top perspective view of the smartwatch of FIG. 4A, with the elastomeric fitting body of FIGS. 3A-3C fitted thereover.

FIG. 4D is a bottom perspective view of the smartwatch of FIG. 4C, with the elastomeric body fitted on the smartwatch with a body-facing contact.

FIG. 4E is a cross section along Line A-A of FIG. 4C, illustrating the fitting body in situ on the smartwatch.

FIG. 5A is an exploded view of one embodiment of the fitting body as a metal clip and plastic cover.

FIG. 5B is an exploded view of the components of FIG. 5A from the side.

FIG. 5C is top view of the fitting body of FIGS. 5A and 5B fitted upon a smartwatch, illustrating the conductive conduit of the fitting body.

FIG. 5D is the top view of the fitting body of FIGS. 5A-C illustrating the plastic cover fitted over the metal clip.

FIG. 5E is a bottom perspective view illustrating the body contact of the smartwatch shown in FIGS. 5E and 5D, illustrated here as having four conductive points for the body contact.

FIG. 5F is a top perspective view of the smartwatch in FIGS. 5C-5E with the fitting body attached to the smartwatch and the fitting body contact is held to the wrist of the wearer.

FIG. 6 is a graph of two simultaneous recordings to demonstrate the advantages of a single-wrist recording to detect the EMG signal associated with respiratory effort.

FIG. 7 is a perspective view of one embodiment of the device comprised of braided wire and beads that can fit on the wrist of an infant.

FIG. 8A is a perspective view of the device body embodied as a three-wire braid with conductive tab contacting each wire in two places.

FIG. 8B is the device body of FIG. 8A illustrating wrapped contacts for each of the three electrodes, as well as a connective contact for the transmitter.

FIG. 8C is the device body of FIGS. 8A, 8B illustrating the beads placed over one of the connective contacts for detection of bioelectric signals, with three contacts exposed that will be in conductive contact with a transmitter.

FIG. 9 is one embodiment of the device comprised of a braided wired with beads that detect bioelectrical signals of an infant and a transmitter sends the detected signals to a receiver.

### DETAILED DESCRIPTION OF THE INVENTION

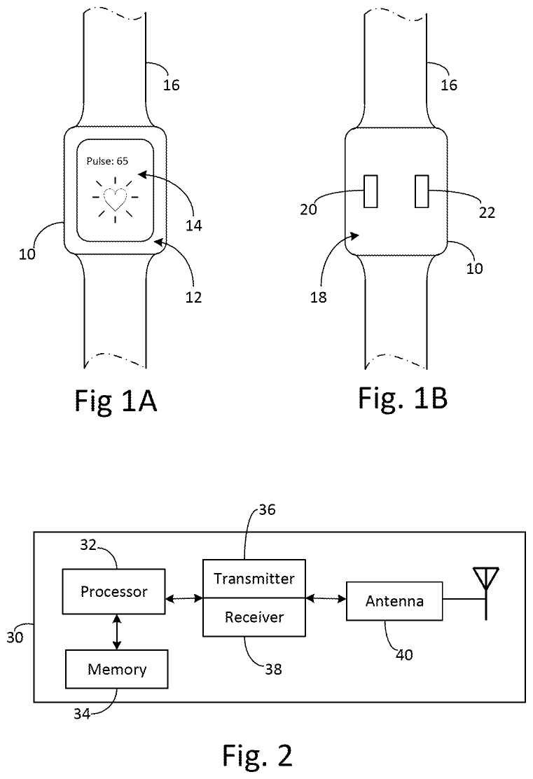

With reference to the figures in which like numerals represent like elements throughout the several views, FIG. 1A is a top perspective view of one embodiment of a wearable device 10 embodied as a smartwatch. FIG. 1B is a bottom perspective view of the wearable device 10 of FIG. 1A illustrating two body-contacting electrical contacts 18 and that can monitor the bioelectrical signals of the body. FIG. 2 is a block diagram of the computer platform 30 and components thereon which is within the body of the device illustrated in FIGS. 1A and 1B and 4A and 4B.

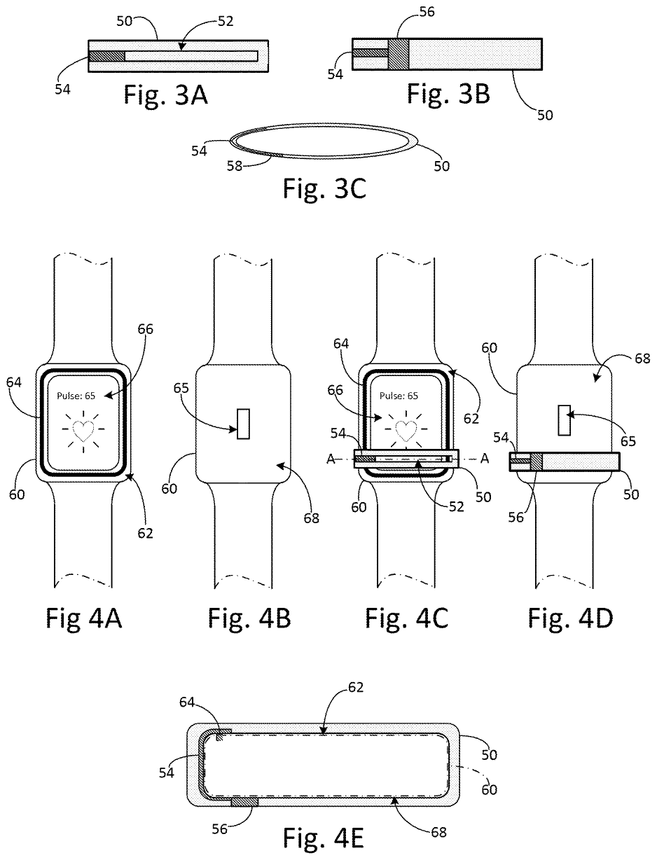

As shown in FIGS. 1A, 1B and 2, the present invention provides a device and method for monitoring electromyographic signals at a wearable health-data device (such as device 10) that selectively monitors bioelectrical signals of a human body, such as an existing smartwatch or other wireless biosensor. As is shown in FIGS. 3A-5F, the present invention can include modifying an existing health-data device, such as smartwatch 60 in FIGS. 4A-4E with one body-facing contact 70, can also be modified with a fitting body 50 that allows dual contacts (contact 65 and fitting body contact 56) to be simultaneously held against the human body, preferably one a limb such as a wrist, such that electromyographic signals can be monitored.

In one embodiment, the device for monitoring electromyographic signals includes a device body 10 having a surface 18 thereof that is configured to be held against a human body. There are two electrical contacts 20,22 on the surface 18 of the device body 10, with each contact placed apart from the other on the device body 10 such that each contact is conductively held against the human body such that a bioelectrical electrical signal is passed between the two electrical contacts 20,22.

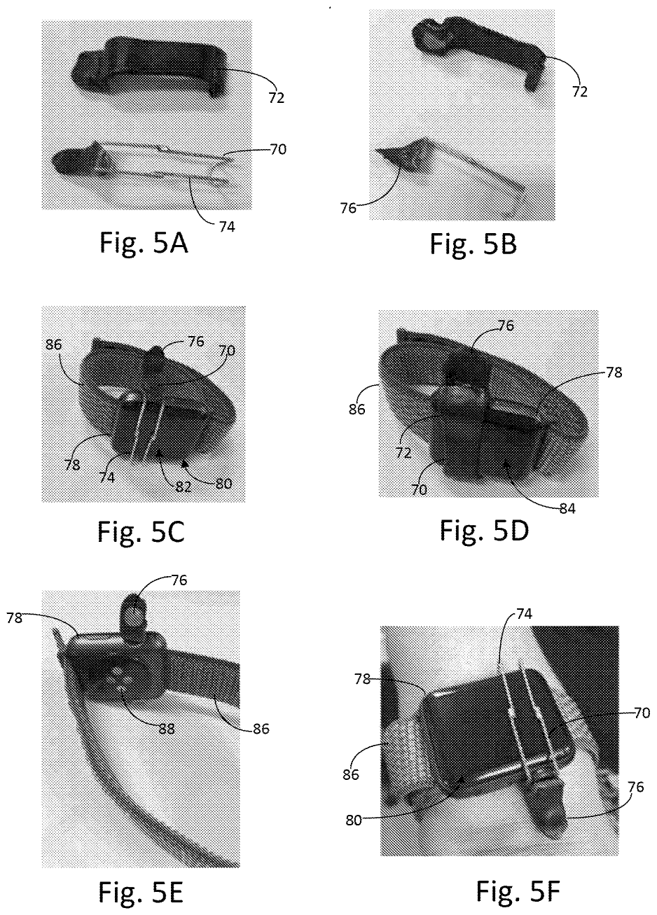

FIG. 1A is a top perspective view of one embodiment of a wearable device embodied as a smartwatch. The device body 10 has a top surface 12 that includes a visual display 14 for the user. The device body 10, in this embodiment includes a wristband 16 that holds the device body 10 against a wrist (as is shown in FIG. 5F) such that the contacts 20,22 are held against the wrist.

FIG. 1B is a bottom perspective view of the smartwatch of FIG. 1A specifically illustrating the two body-contacting electrical contacts 20,22 that can monitor the EMG signals of the body. The device body 10 includes the bottom surface 18 with the contacts 20,22 physically accessible thereat. The spacing between the contacts 20,22 should be enough to allow a biological electrical signal to be differentially recorded therebetween, so approximately a spacing of 0.3 cm or greater.

FIG. 2 is a block diagram of the computer platform 30 and components thereon which is within the device body 10, illustrated in FIGS. 1A and 1B, and device body 60 in FIGS. 4A and 4B. On the computer platform 30 is a processor 32 and memory 34 that can execute select programs for the smartwatch. The device body 10 can further be embodied as including a transmitter 36 therein that in communication with the processor 32 across the computer platform 30 and selectively transmits the respiratory effort data output from the computer platform 30 through an antenna 40. Furthermore, the device body 10 can further include a receiver 38 therein that is in communication with the processor 32 across the computer platform 30, and is in communication with a communication network through the antenna 40, such as a Wifi or Bluetooth network, and selectively receives data sent thereacross and relays the data to the processor 32. In such embodiment the processor 32 can thus send data and receive software updates and update resident software on the processor 32 as well as data held in memory 34.

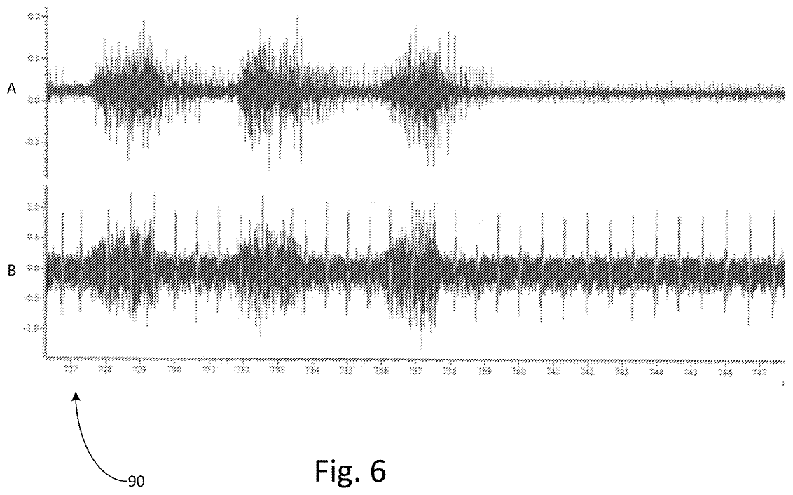

The computer platform 32 can thus be configured to selectively monitor bioelectrical signals between the electrical contacts 20,22 on the device body 10, isolate one or more electromyographic signals within the monitored bioelectrical signals (such as shown in FIG. 6), compile respiratory effort data from the one or more electromyographic signals, and selectively output the respiratory effort data, such as on display 14 or potentially as data through the transmitter 36. Thus, through the processing on the computer platform 30, the device can determine the presence of apnea or a respiratory seizure based upon the respiratory effort data based upon the respiratory effort data from the EMG signals that can be obtained from the bioelectrical signals across the two contacts 20,22.

As shown in FIGS. 3A-3C and 4A-4E, in one embodiment, the invention includes a fitting for modifying a wearable health-data device to monitor electromyographic signals, such as a smartwatch 60 or other device that does not have adjacent body-contacting electrodes or electrical contacts such that EMG signals are more easily discerned from ECG signals. FIG. 3A is a top view of one embodiment of a fitting body 50 comprised of an elastomer that modifies a smartwatch 60 which has only one body-facing electrical contact 65.

With reference to FIG. 3A, the fitting body 50 includes an electrical contact 56 and electrical conduit 54, with a mechanism of elastomeric deformation that allows the selectively attachment of the fitting body 50 to a health-data device, such as smartwatch 60 shown in FIGS. 4A-4E, that is configured to be selectively held against a human body. FIG. 3A shows the open portion 52 that allows the display 66 to be view when fitted on smartwatch 60, and electrical conduit 54 that travels up to touch the conductive crown 64 on the smartwatch 60.

FIG. 3B is a bottom view of the fitting body 50 of FIG. 3A, illustrating the electrical contact 56 and electrical conduit 54. FIG. 3C is a sideview of the fitting body 50 of FIGS. 3A and 3B. In FIG. 3C, the elastomeric fitting body 50 is unstretched and not yet fitted on smartwatch 60. Alternatively, the electrical conduit 54 can be a wire, or the electrical contact 56 and electrical conduit 54 of the fitting body 50 can be the same conductive component, such as a single conductive wire or other contiguous conductive element.

FIG. 4A is a top perspective view of a smartwatch 60 having a conductive crown 64. The smartwatch 60, such as an Apple watch, includes a first electrical contact 65 that is selectively conductively held against the human body and a second electrical contact, such as conductive crown 64, that is not conductively held against the human body. The fitting body 50 can further be configured to be selectively attached to the body of the smartwatch 60 such that the electrical contact 56 of the fitting body 50 is conductively held against the human body when the smartwatch 60 is held thereagainst, with the fitting body 50 further attached such that a bioelectrical signal is passed from the electrical contact 56 of the fitting body 50 through the electrical conduit 54 to the second electrical contact, such as conductive crown 64.

FIG. 4B is a bottom perspective view of the smartwatch of FIG. 4A illustrating a single body-facing contact 65 on the bottom surface 68 of smartwatch 60. FIG. 4C is a top perspective view of the smartwatch of FIG. 4A, with the elastomeric fitting body 50 of FIGS. 3A-3C fitted thereover. The fitting body 50 is in elastomeric deformation and fitted over the body of the smartwatch, as can be seen in FIG. 4E, which is a cross section along Line A-A of FIG. 4C, illustrating the fitting body 50 in situ on the smartwatch. The fitting body 50 is fitted such that open portion 52 is over the display 66 so that the user can view the display 66. The electrical conduit 54 conductively touches the conductive crown 64 and bridges an electrical connection to electrical contact 56.

FIG. 4D is a bottom perspective view of the smartwatch of FIG. 4C, with the elastomeric fitting body 50 fitted on the smartwatch 50 with a body-facing contact 56 adjacent the body-facing contract 65 on the bottom surface 68 of the smartwatch 60. FIG. 4E, which is a cross section along Line A-A of FIG. 4C, illustrates the elastomeric fitting body 50 in situ on the smartwatch 60, deformed about the body of the smartwatch 60 with the fitting electrical contract 56 on the bottom surface 68 of the smartwatch 60. The electrical conduit 54 extends around the side of the smartwatch 60 to conductively contact the conductive crown 64.

FIG. 5A-5F illustrate one embodiment of the fitting body 70 as a metal clip 74 and plastic cover 72. In one embodiment, the metal clip 74 that snaps to the face 82 of a smartwatch 78 that has an electrically conductive crown and provides an electrical contact 76 that redirects the smartwatch 78 crown electrode contact onto the skin of the arm wearing the watch, as shown in FIG. 5F. The electrical contact of the metal clip 74 that touches the crown is electrically connected through the metal clip 74 of the “right-angled” assembly to the electrical contact 76 directed toward the skin surface. In this embodiment, this also prevents the watch from being used for two-handed recordings while the accessory is in place. The position of the electrical surface contacting the watch crown can be fixed in relation to the position of the electrical contact 76 that will touch skin, or can be adjusted perpendicularly to the face of the watch to optimize skin contact.

In another embodiment, the position of the electrical contact (metal clip 74) with the crown is fixed, and a flexible (e.g., spring-loaded) contact reaches from the watch accessory to the skin to optimize skin contact.

FIG. 5B is an exploded view of the components of FIG. 5A from the side, i.e. the metal clip 74 and plastic cover 72, illustrating the electrical contact 76 for the body. FIG. 5C is top view of the fitting body of FIGS. 5A and 5B fitted upon a smartwatch 78, illustrating the conductive conduit (metal clip 74) of the fitting body 70 covering the face 80 of the smartwatch 78 that also includes a display 82. The smartwatch 78 also includes a wristband to hold the smartwatch to a wrist as shown in FIG. 5F.

FIG. 5D is a top view of the fitting body of FIGS. 5A-C illustrating the plastic cover 72 fitted over the metal clip 74. The plastic cover 72 fits over electrical contract 76 and insulates the bioelectric signal from the conductive crown to the electrical contact 76.

FIG. 5E is a bottom perspective view of the smartwatch 78 illustrating the electrical contact 76 of the smartwatch shown in FIGS. 5A-5D, with the body contact 88 of the smartwatch illustrated here as having four conductive points for the body contact. FIG. 5F is a top perspective view of the smartwatch 78 in FIGS. 5C-5E, with the fitting body 70 attached to the smartwatch and the fitting body contact 76 is held to the wrist of the wearer. The metal clip 74 is attached across the face 80 of the smartwatch 78 and the plastic cover 72 has not yet been fitted thereover.

FIG. 6 is a graph 90 of two simultaneous recordings to demonstrate the advantages of single wrist recording to detect the EMG signal associated with respiratory effort. Both recordings were taken with 10 Hz to 1 kHz filtering and 1000× gain digitized at 2000 Hz. In the top signal A, both electrodes were on the left wrist. In the bottom signal B, one electrode was on each wrist. The ground electrode was shared and was also located on the left wrist. The y-axis shows units of millivolts for both channels and the x-axis is time in seconds.

What is shown is three instances of the Mueller maneuver, an attempt to inspire without moving air, followed by a period of resting activity. The intense effort associated with each inspiratory attempt is associated with a dramatic increase in EMG activity, shown in top signal A. The contrast between the respiratory effort-associated EMG and the ECG signal is clear in the bottom signal B, which contains the ECG. Especially prominent is the absence of ECG from the one-hand recording shown in the top signal A. This recording will also greatly simplify the processing demands that may be used to quantify such signals.

In the smartwatch-modified-with-fitting configuration, the invention can therefore include the device body 10 which has a surface 18 thereof, where the device body 10 configured to be held against a human body, and a first electrical contact 65 that is selectively conductively held against the human body and a second electrical contact (conductive crown 64) that is not conductively held against the human body. The fitting body 50 has a fitting electrical contact 56 and fitting electrical conduit 54, and a mechanism selectively attaching the fitting body 50 to the device such that the fitting electrical contact 56 is conductively held against the human body and a bioelectrical signal is passed from the fitting electrical contact and through the fitting electrical conduit 54 to the second electrical contact (conductive crown 64) of the device. Included in the device body 50 is a computer platform 30 that is configured to selectively monitor bioelectrical signals between the fitting electrical contact 56 and second electrical contact 64, isolate one or more electromyographic signals within the monitored bioelectrical signals, compile respiratory effort data from the one or more electromyographic signals, and selectively output the respiratory effort data.

As shown in FIGS. 1A, 1B and 2, the invention also includes a method for monitoring electromyographic signals at a wearable device holding a device against a human body, the device having a device body 10 with a surface 18 thereof and two electrical contacts 20,22 on the surface of the device body 10, with each contact 20,22 placed apart from the other on the device body 10 such that each contact is conductively held against the human body such that a bioelectrical electrical signal is passed between the two electrical contacts 20,22. Then the method includes the steps of selectively monitoring bioelectrical signals between the electrical contacts 20,22 on the device body 10, isolating one or more electromyographic signals within the monitored bioelectrical signals, compiling respiratory effort data from the one or more electromyographic signals, and selectively outputting the respiratory effort data.

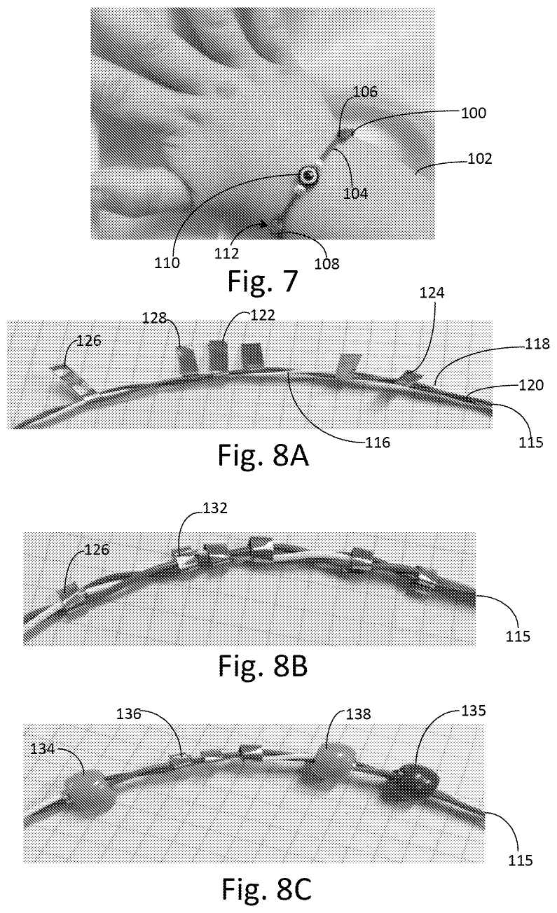

FIG. 7 is a perspective view of one embodiment of the device 100 for monitoring electromyographic signals, particularly on the arm 102 of an infant. The device 100 has a flexible device body 104, with the body 104 comprised of a plurality of electrical wires (such as wires 116,118,120 in FIG. 8A), with at least two conductive electrodes 106,108 on the body 100 that each have fully conductive exterior surfaces 112 to contact skin continuously, and each electrode 106,108 having a fully conductive interior surface that contacts the exposed surface at least one of the plurality of electrical wires 116, 118, 120 of the body 100. The device 100 also has a computer platform 110 on the body 100, the computer platform 110 configured to selectively monitor bioelectrical signals between the electrodes 106,108 on the device body 100, isolate one or more electromyographic signals within the monitored bioelectrical signals, compile respiratory effort data from the one or more electromyographic signals, and selectively transmit the respiratory effort data.

As shown in the perspective view of FIG. 8A, the device body 115 can be embodied as a three-wire braid (wires 116, 118, 120) with each having a conductive tab contacting each wire in two places, e.g. tabs 122, 124 of wire 120. The three-wire braid forms the core of the body 115 to create a bracelet. Each wire is exposed and contacts a conductive tab (e.g. tabs 122, 124) in two places. For example, the white wire 116 above shows a single contact tab 126 at the far left and the first of 3 contact tabs 128 in the center. The three center contacts serve the controller 110 (FIG. 7). The lateral contacts on the left and right of the controller 110 will be electrode contacts 106, 108. Thus, the flexible device body 100 can be comprised of braided wire and beads that can fit on the wrist 102 of an infant.

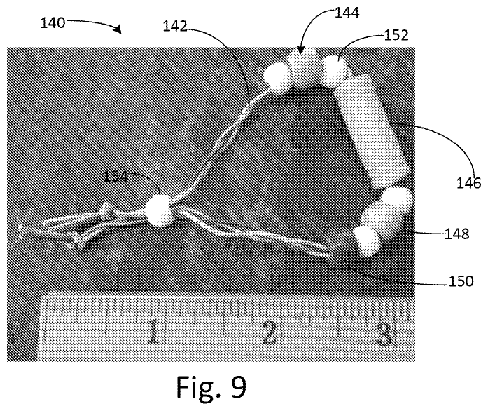

The computer platform 110 can include a recording amplifier, Bluetooth transmitter, and power source. As shown further in FIG. 9, the device body 140 can contain a fourth bead 146 that contains a miniature recording amplifier (optional digitization hardware), Bluetooth transmitter and small battery.

FIG. 8B is the device body 115 of FIG. 8A illustrating wrapped contacts, e.g. contact tab 126, 132 for white wire 116, for each of the three electrodes, as well as a connective contact (140 in FIG. 8C) for the transmitter 146 (FIG. 9). FIG. 8C is the device body 115 of FIGS. 8A, 8B illustrating the beads 134, 135, 138 placed over one of the connective contacts, e.g. bead 134 over conductive contact 126 in FIG. 8B, for detection of bioelectric signals, with three contacts 136 exposed that will be in conductive contact with a transmitter 146 (FIG. 9). The wrapped contacts 136 will create three electrodes, but not the control unit 110 (FIG. 7) are shown with their respective electrodes. For construction of the wire, copper can be used, but a stiffer metal (e.g. steel) can be configured to have a continuous spring-like contact with the center of the electrode if desired.

FIG. 9 is one embodiment of the device 140 comprised of a braided wired 142 with beads 144, 148, 150, that detect bioelectrical signals of an infant and a transmitter 146 sends the detected signals to a receiver (not shown). This embodiment uses plastic beads to simulate the three electrodes two gray beads 144, 148 for the primary recording electrodes and one green bead 150 for an optional ground electrode). The large green bead 146 is also the control unit for the bracelet 142. The white beads 152 serve as locking spacers that lock the position of the recording beads 144,148 relative to the control unit and lock the total length of the bracelet 142 once adjusted. The braided 3-color wires serve to connect each bead (one color for each electrode bead) to the control unit (transmitter 146). The colored insulation is stripped for each wire under its designated bead such that each conductive bead contacts only one wire, as is more clearly shown in FIGS. 8A-8C.

In one embodiment of the design, the conductive beads 144, 148 are spaced using non-conductive, smaller outside diameter spacers 152 to separate the beads from one another. In this figure, the spacer beads 152 are white and the electrode beads are gray 144, 148 or green 150. The bracelet 142 itself is adjusted to the proper length by a small clip (shown as a white bead 154 near the two ends of the bracelet) that holds the bracelet at the right length. Other mechanical adjustment devices for the bracelet 142 would be apparent to one of skill in the art. In one embodiment, the telemetry/control bead (transmitter 146) signals a telemetry antenna in exactly the same way that small extant telemetry units work. The telemetry antenna (not shown) can be hung on the side of a crib or placed beneath the crib mattress.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A device for monitoring electromyographic signals, comprising:
a device body having a surface thereof, the device body configured to be held against a human body;
two electrical contacts on the surface of the device body, each contact placed apart from each other on the device body such that each contact is conductively held against the human body upon the device body being held against the human body such that a bioelectrical electrical signal is passed between the two electrical contacts; and
a computer platform contained within the device body, the computer platform configured to:
selectively monitor bioelectrical signals between the electrical contacts on the device body;
isolate one or more electromyographic signals within the monitored bioelectrical signals;
compile respiratory effort data from the one or more electromyographic signals; and
selectively output the respiratory effort data.

2. The device of claim 1, wherein the device body further including a transmitter therein, the transmitter in communication with the computer platform and selectively transmitting the respiratory effort data output from the computer platform.

3. The device of claim 2, wherein the device body further including a receiver therein, the receiver in communication with the computer platform, the receiver further in communication with a communication network and selectively receiving data sent thereacross.

4. The device of claim 1, wherein the device body is configured to be attached to a wristband such that the wristband selectively holds the device body and the electrical contacts against a wrist.

5. The device of claim 1, wherein the computer platform further configured to determine a presence of apnea based upon the respiratory effort data.

6. The device of claim 1, wherein the computer platform further configured to determine a presence of respiratory arrest based upon the respiratory effort data.

7. A fitting for modifying a wearable health-data device to monitor electromyographic signals, comprising:
a body including an electrical contact and electrical conduit;
a mechanism for selectively attaching the body to a health-data device that is configured to be selectively held against a human body, the health-data device including a first electrical contact that is selectively conductively held against the human body and a second electrical contact that is not conductively held against the human body; and
the fitting further configured to be selectively attached to the body of the health-data device such that the electrical contact of the fitting is conductively held against the human body when the health-data device is held thereagainst, the fitting further attached such that a bioelectrical signal is passed from the electrical contact of the fitting and through the electrical conduit to the second electrical contact of the health-data device.

8. The fitting of claim 7, wherein the mechanism for selectively attaching the body to a health-data device is a mechanical clip.

9. The fitting of claim 7, wherein the mechanism for selectively attaching the body to a health-data device is an elastomeric mechanism.

10. The fitting of claim 7, wherein the electrical conduit is a wire.

11. The fitting of claim 7, wherein the electrical contact and electrical conduit of the fitting are a same conductive component.

12. A device for monitoring electromyographic signals, comprising:
a device body including:
a surface thereof, the device body configured to be held against a human body;
a first electrical contact that is selectively conductively held against the human body;
a second electrical contact that is not conductively held against the human body;
a fitting including:
a fitting body having a fitting electrical contact and fitting electrical conduit; and
a mechanism selectively attaching the fitting body to the device body such that the fitting electrical contact is conductively held against the human body and a bioelectrical signal is passed from the fitting electrical contact and through the fitting electrical conduit to the second electrical contact of the device; and
a computer platform contained within the device body, the computer platform configured to:
selectively monitor bioelectrical signals between the fitting electrical contact and second electrical contact;
isolate one or more electromyographic signals within the monitored bioelectrical signals;
compile respiratory effort data from the one or more electromyographic signals; and
selectively output the respiratory effort data.

13. The device of claim 12, wherein the device body further including a transmitter therein, the transmitter in communication with the computer platform and selectively transmitting the respiratory effort data output from the computer platform.

14. The device of claim 12, wherein the device body further including a receiver therein, the receiver in communication with the computer platform, the receiver further in communication with a communication network and selectively receiving data sent thereacross.

15. The device of claim 12, wherein the computer platform further configured to determine a presence of apnea based upon the respiratory effort data.

16. The device of claim 12, wherein the computer platform further configured to determine a presence of a seizure based upon the respiratory effort data.

17. A method for monitoring electromyographic signals at a wearable device, comprising:
holding a device against a human body, the device having a device body with a surface thereof and two electrical contacts on the surface of the device body, each contact placed apart from each other on the device body such that each contact is conductively held against the human body such that a bioelectrical electrical signal is passed between the two electrical contacts;
selectively monitoring bioelectrical signals between the electrical contacts on the device body;
isolating one or more electromyographic signals within the monitored bioelectrical signals;
compiling respiratory effort data from the one or more electromyographic signals; and
selectively outputting the respiratory effort data.

18. The method of claim 17, wherein the device body further including a transmitter therein, and further including selectively transmitting the respiratory effort data output from the device.

19. The method of claim 18, wherein the device body further including a receiver therein, the receiver further in communication with a communication network, and further including selectively receiving data sent thereacross.

20. The method of claim 17, wherein holding a device against a human body is holding the device with a wristband such that the wristband selectively holds the device body and the electrical contacts against a wrist.

21. The method of claim 17, further including determining a presence of apnea based upon the respiratory effort data.

22. The method of claim 17, further including determining a presence of a seizure based upon the respiratory effort data.

23. A method of monitoring electromyographic signals by modifying a wearable health-data device with a fitting, comprising:
selectively attaching a fitting body to a health-data device, the health-data device configured to be selectively held against a human body, the health-data device including a first electrical contact that is selectively conductively held against the human body and a second electrical contact that is not conductively held against the human body, and the fitting body including an electrical contact and electrical conduit;
wherein the fitting body further selectively attached to the health-data device such that the electrical contact of the fitting is conductively held against the human body and a bioelectrical signal is passed from the electrical contact of the fitting and through the electrical conduit to the second electrical contact of the health-data device;
selectively monitoring bioelectrical signals between the fitting electrical contact and second electrical contact;
isolating one or more electromyographic signals within the monitored bioelectrical signals;
compiling respiratory effort data from the one or more electromyographic signals; and
selectively outputting the respiratory effort data.

24. The method of claim 23, wherein selectively attaching the fitting body to a health-data device is selectively attached the fitting body with a mechanical clip.

25. The method of claim 23, wherein selectively attaching the fitting body to a health-data device is selectively attaching the fitting body with an elastomeric mechanism.

26. The method of claim 23, further including determining a presence of apnea based upon the respiratory effort data.

27. The method of claim 23, further including determining a presence of respiratory arrest based upon the respiratory effort data.

28. A device for monitoring electromyographic signals, comprising:
a flexible device body, the body comprised of a plurality of electrical wires;
at least two conductive electrodes on the body that each have fully conductive exterior surfaces to contact skin continuously, each electrode having a fully conductive interior surface that contacts an exposed surface of at least one of the plurality of electrical wires of the body; and
a computer platform on the body, the computer platform configured to:
selectively monitor bioelectrical signals between the electrodes on the device body;
isolate one or more electromyographic signals within the monitored bioelectrical signals;
compile respiratory effort data from the one or more electromyographic signals; and
selectively transmit the respiratory effort data.

29. The device of claim 28, wherein:
the flexible device body is comprised of a braided 3-wire bracelet, each having at least one exposed conductive portion; and
the conductive electrodes are three beads with fully conductive interior surfaces that contact at least one exposed surface of each of one of the three wires.

30. The device of claim 29, wherein the computer platform further including a recording amplifier, Bluetooth transmitter and power source.

31. A method for monitoring electromyographic signals at a wearable device, comprising:
holding a flexible device body against a single limb of a human, the body comprised of a plurality of electrical wires with at least two conductive electrodes on the body that each have fully conductive exterior surfaces to contact skin continuously, each electrode having a fully conductive interior surface that contacts an exposed surface of at least one of the plurality of electrical wires of the body;
selectively monitoring bioelectrical signals between the electrodes on the device body;
isolating one or more electromyographic signals within the monitored bioelectrical signals;
compiling respiratory effort data from the one or more electromyographic signals; and
selectively transmitting the respiratory effort data.

32. The method of claim 31, wherein the device body further including a transmitter therein, and further including selectively transmitting a respiratory effort data output from the device body.

33. The method of claim 31, wherein the device body further including a receiver therein, the receiver further in communication with a communication network, and further including selectively receiving data at the receiver.

34. The method of claim 31, wherein holding a device against a human body is holding a flexible bracelet against a wrist.
